(19) United States
(12) Plant Patent Application Publication
(10) Pub. No.: US 2002/0174470 P1
(43) Pub. Date: Nov. 21, 2002

(54) Dahlia plant named 'Dapahapu'

(75) Inventor: Francois Van Hee, Emkhuizen (NL)

Correspondence Address: ROCKEY, MILNAMOW & KATZ, LTD., Two Prudential Plaza, 180 North Stetson, Suite 4700, Chicago, IL, 60601, US

(73) Assignee: Ball Floraplant, a division of Ball Horticultural Company

(21) Appl. No.: 09/819,315

(22) Filed: Mar. 28, 2001

(51) Int. Cl. A01H 5/00

(52) U.S. Cl. PLT/321

(57) ABSTRACT

A new and distinct Dahlia plant named Dapahapu characterized by its unique purple and white flower color, medium growth habit and simple leaf arrangement.

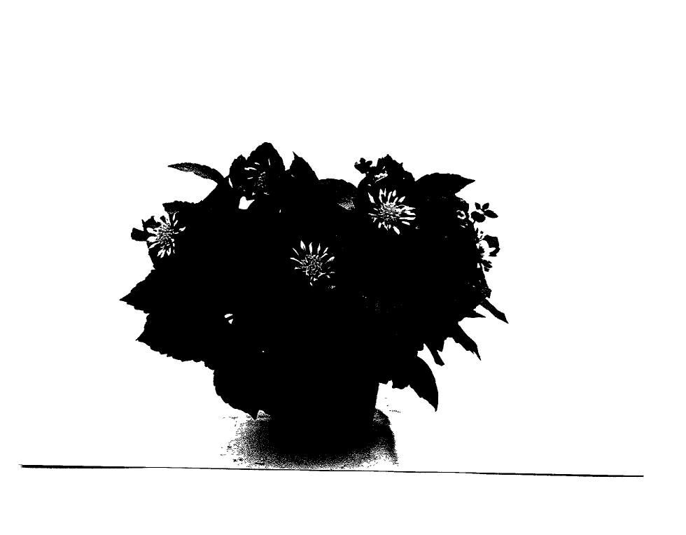

### BACKGROUND OF THE INVENTION

The present invention comprises a new and distinct Dahlia plant, botanically known as Dahlia variabills and hereinafter referred to by the cultivar name Dapahapu. The breeding program which resulted in the production of the new cultivar was carried out in Enkhuizen, The Netherlands. The female or seed parent of Dapahapu was a proprietary Dahlia plant designated H122. The male or pollen parent of Dapahapu was a proprietary Dahlia plant designated DA2.

Asexual reproduction of the new cultivar by terminal or stem cuttings taken during 2000 at West Chicago Ill., USA has demonstrated that the characteristics of the new cultivar as herein described are firmly fixed and are retained through successive generations of such asexual propagation.

### SUMMARY OFTHE INVENTION

It was found that the cultivar of the present invention:

(a) exhibits an upright mounded habit,

(b) forms purple and white ray florets and

(c) exhibits a compact to moderate growth habit.

Dapahapu has not been observed under all possible environmental conditions to date. Accordingly, it is possible that the phenotype may vary somewhat with variations in the environment, such as temperature, light intensity, and day length.

The new cultivar of the present invention can be compared to Lizzy (U.S. Plant Pat. No. 9,846). However, in side by side comparisons, Dapahapu exhibits shorter internodes, smaller inflorescences and ray florets of a deeper purple as well as some purple and white ray florets as detailed in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plant was grown for nine weeks in a greenhouse at West Chicago, Ill., USA.

### DETAILED BOTANICAL DESCRIPTION

The chart used in the identification of colors described herein is the R.H.S Colour Chart of The Royal Horticultural Society, London, England. The color values were determined on Mar. 6, 2001 in West Chicago, Ill. The readings were taken between 10:00 and 11:45 p.m. under natural light conditions. The plants used for the following were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night. Plants were pinched three weeks after the planting of rooted cuttings. The following measurements and descriptions were made eight weeks after planting.

Botanical Classification: Dahlia variabilis cultivar Dapahapu

Parentage:

Female parent.—Proprietary Dahlia plant designated H122 (not patented)

Male parent.—Proprietary Dahlia plant designated DA2 (not patented)

Propagation:

Type cutting.—Terminal tip.

Time to initiate roots.—Approximately 7 -10 days.

Time to develop roots.—Approximately 28 -34 days.

Root description.—Fibrous and well branched.

Tubers.—Will form under short day conditions of at least 13 to 14 hours of darkness.

Plant Description:

General appearance and form.—Upright and mounded, moderatly compact.

Height above pot.—Approximately 10 cm.

Width.—Approximately 15 cm.

Branching habit.—Freely branching. Pinching helps to initiate branching.

Branch description.—Diameter: Approximately 6 mm. Texture: Smooth. Color: Between 143A and 146A with moderate anthocyanin coloration at nodes. Internode length: Approximately 1.5 cm.

Foliage Description

Form.—Mature foliage is single.

Shape.—Ovate.

Margin.—Serrate.

Apex.—Acuminate.

Base.—Attenuate.

Arrangement.—Opposite.

Surface.—Upper surface slightly rugose.

Color of mature foliage.—Upper surface: Closest to 137A with veins of 146B.

Color of mature foliage.—Lower surface: Closest to 147B with veins of 146B.

Size.—Mature leaves are approximately 6.6 cm in length, approximately 4.6 cm in width.

Petiole.—Approximately 3.5 cm in length, 3 mm in width and 144A in color.

Flower Description

Outdoor Flowering Habit.—Dapahapu is freely flowering under outdoor growing conditions with substantially continuous blooming from spring through fall.

Appearance.—Double, arising from leaf axils on strong peduncles. Disc and ray florets arranged acropetally on a capitulum

Inflorescence diameter.—Approximately 6.8 cm. Disc diameter: Approximately 1.8 cm

Ray florets.—Quantity: Approximately 30. Shape: Elongated, cupped. Length: Approximately 2 cm. Apex: Pointed to slightly rounded. Base: Attenuate. Margin: Entire. Texture: Smooth. Color: Outer whorls 187B, inner whorl is 155D.

Disc florets.—Shape: Elongated, cylindrical. Length: 1 cm. Diameter: 2 mm. Color: 15B. Involucral bracts.—Quantity: 5. Shape: Diamond shaped. Tip: Broadly acute. Margin: Entire. Texture: Smooth. Color: 137A

Reproductive Organs.—Androecium.—On dics florets. Anthers approximately 3 mmin length, closest to 15A in color. Gynoecium.—On ray and disc florets. Pistil length: Approximately 1 cm . Pollen color is 21B.

Seed Production: Seed production has not been observed.

Disease Resistance: Disease resistance has not been observed.

TABLE A

| CHARACTERISTIC | DAPAHAPU | LIZZY |
|---|---|---|
| FULLY OPENED OUTER RAY | 187A | 60A |
| FLORETS | | |
| INFLORESCENCE | 6.8 | 9 |
| DIAMETER-CM | | |
| INTERNODE LENGTH-CM | 1.5 | 2.5 |

I claim:

1. A new and distinct cultivar of Dahlia named Dapahapu substantially as herein shown and described, which:
(a) exhibits and upright mounded habit,
(b) forms purple and white ray florets and
(c) exhibits a moderate growth habit.
